(19) United States
(12) Patent Application Publication
(10) Pub. No.: US 2021/0375473 A1
(43) Pub. Date: Dec. 2, 2021

(54) SYSTEMS AND METHODS FOR HYPERTENSION MONITORING

(71) Applicant: Apple Inc., Cupertino, CA (US)

(72) Inventors: Leon Alexander GATYS, Seattle, WA (US); Gleb KICHAEV, La Mesa, CA (US); Charles H. GREENBERG, South San Francisco, CA (US); Charles Graham Haver CRISSMAN, Issaquah, WA (US); Christopher J. BROUSE, Cupertino, CA (US)

(21) Appl. No.: 17/336,188

(22) Filed: Jun. 1, 2021

Related U.S. Application Data
(60) Provisional application No. 63/033,802, filed on Jun. 2, 2020.
(60) Provisional application No. 63/146,536, filed on Feb. 5, 2021.

(51) Int. Cl. G16H 50/30; G16H 50/20; A61B 5/00; A61B 5/0225

(52) U.S. Cl. CPC G16H 50/30; G16H 50/20; A61B 5/6801; A61B 5/02255; A61B 5/7253

(57) ABSTRACT

A wearable device can be used for hypertension monitoring. The wearable device can include a motion sensor and an optical sensor. The data from the sensors can be processed in the wearable device and/or by another device in communication with the wearable device to provide an early screening for undiagnosed hypertension. If the screening estimates undiagnosed hypertension for a user, the user can then be notified to seek a proper hypertension diagnosis. The hypertension monitoring can include a first stage to estimate one or more short-term hypertension scores or parameters. The hypertension monitoring can also include a second stage to estimate a long-term hypertension score using accumulated short-term scores/parameters to estimate hypertension.

### CROSS-REFERENCE TO RELATED APPLICATIONS

This application claims the benefit of U.S. Provisional Application No. 63/033,802, filed Jun. 2, 2020, and U.S. Provisional Application No. 63/146,536, filed Feb. 5, 2021, the contents of which are incorporated herein by reference in their entirety for all purposes.

### FIELD

This relates generally to systems and methods for hypertension monitoring, and more particularly, to hypertension monitoring using a wearable device.

### BACKGROUND

Without proper diagnosis and treatment, hypertension (high blood pressure) can increase risk of health problems, such as stroke and heart attack. Hypertension often goes undetected because symptoms may not manifest for months or years. Even without symptoms, however, hypertension can damage the heart and blood vessels. Accordingly, providing users with an indication of hypertension can be useful to improve health.

### SUMMARY

This relates to systems and methods for monitoring for hypertension using a wearable device. The wearable device can include a motion and/or orientation sensor (e.g., accelerometer, gyroscope, inertia-measurement unit (IMU), etc.) and an optical sensor. The data from the sensors can be processed in the wearable device and/or by another device in communication with the wearable device to provide an early screening for undiagnosed hypertension. If the screening estimates undiagnosed hypertension for a user, the user can then be notified to seek a proper hypertension diagnosis. The hypertension monitoring can include a first stage to estimate one or more short-term hypertension scores or parameters. The short-term hypertension scores/parameters can be correlated with blood pressure. In some examples, the short-term hypertension scores/parameters can include a systolic blood pressure score (or parameters) and a diastolic blood pressure score (or parameters). The hypertension monitoring can also include a second stage to estimate a long-term hypertension score using accumulated short-term scores/parameters (e.g., for a threshold period of time or a threshold number of short-term hypertension scores/parameters) to estimate hypertension.

### BRIEF DESCRIPTION OF THE DRAWINGS

FIGS. 1A-1B illustrate an example system that can be used for hypertension monitoring according to examples of the disclosure.

FIG. 2 illustrates an example block diagram for hypertension monitoring according to examples of the disclosure.

FIG. 3. Illustrates and example process for hypertension monitoring according to examples of the disclosure.

FIGS. 4A-4B illustrate example block diagrams of short-term hypertension score generators according to examples of the disclosure.

FIGS. 5A-5C illustrate example block diagrams of long-term hypertension score generators according to examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

This relates to systems and methods for monitoring for hypertension using a wearable device. The wearable device can include a motion and/or orientation sensor (e.g., accelerometer, gyroscope, inertia-measurement unit (IMU), etc.) and an optical sensor. The data from the sensors can be processed in the wearable device and/or by another device in communication with the wearable device to provide an early screening for undiagnosed hypertension. If the screening estimates undiagnosed hypertension for a user, the user can then be notified to seek a proper hypertension diagnosis.

The hypertension monitoring can include a first stage to estimate one or more short-term hypertension scores or parameters. The short-term hypertension scores/parameters can be correlated with blood pressure. In some examples, the short-term hypertension scores/parameters can include a systolic blood pressure score (or parameters) and a diastolic blood pressure score (or parameters). The hypertension monitoring can also include a second stage to estimate a long-term hypertension score using accumulated short-term scores/parameters (e.g., for a threshold period of time or a threshold number of short-term hypertension scores/parameters) to estimate hypertension.

As used herein, “short-term” hypertension scores/parameters can represent hypertension scores/parameters computed from a segment of input data from one or more sensors, each segment corresponding to a first period (e.g., 30 seconds, 1 minute, 2 minutes, 5 minutes, etc.). The short-term hypertension scores/parameters can correlate with the blood pressure for the segment (e.g., of including data acquired in a first period). As used herein, a “long-term” hypertension score can represent a hypertension score computed from input data acquired over the course of a second period (e.g., days, a week, weeks, a month, etc.), which can correlate with the blood pressure for the second period. Thus, “short-term” and “long-term” reflect the relative differences between the first period and second period. The second period used for the “long-term” hypertension score can be orders of magnitude longer than the first period used for the “short-term” hypertension score/parameters.

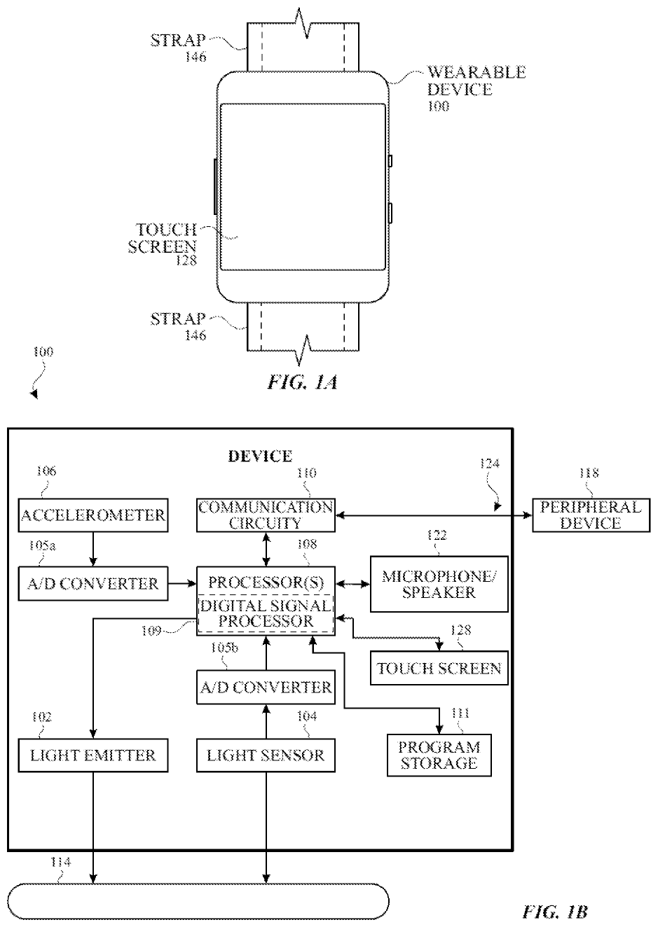

FIGS. 1A-1B illustrate an example system that can be used for hypertension monitoring according to examples of the disclosure. The system can include one or more sensors and processing circuitry to estimate hypertension over a period of time using the data from the one or more sensors. In some examples, the system can be implemented in a wearable device (e.g., wearable device 100). In some examples, the system can implemented in more than one device (e.g., wearable device 100 and a second device in communication with wearable device 100).

FIG. 1A illustrates an example wearable device 100 that can be attached to a user using a strap 146 or other fastener. Wearable device 100 can include one or more sensors used to estimate hypertension over a period of time using the data from the one or more sensors, and optionally can include a touch screen 128 to display the results of hypertension monitoring as described herein.

FIG. 1B illustrates an example block diagram of the architecture of wearable device 100 used to monitor for hypertension according to examples of the disclosure. As illustrated in FIG. 1B, the wearable device 100 can include one or more sensors. For example, the wearable device 100 can optionally include an optical sensor including one or more light emitter(s) 102 (e.g., one or more light emitting diodes (LEDs)) and one or more light sensor(s) 104 (e.g., one or more photodetectors/photodiodes). The one or more light emitters can produce light in ranges corresponding to infrared (IR), green, amber, blue and/or red light, among other possibilities. The optical sensor can be used to emit light into a user's skin 114 and detect reflections of the light back from the skin. The optical sensor measurements by the light sensor(s) can represent a time domain photoplethysmography (PPG) signal. The optical sensor measurements by the light sensor(s) can be converted to digital signals for processing via an analog-to-digital converter (ADC) 105b. The optical sensor and processing of optical signals by the one or more processors 108 can be used, in some examples, for various functions including estimating physiological characteristics (e.g., heart rate, arterial oxygen saturation, etc.), monitoring for physiological conditions (e.g., hypertension), and/or detecting contact with the user (e.g., on-wrist/off-wrist detection).

In some examples, the processing of optical signals by the one or more processors 108 can include identifying cardiac cycles (pulses) in the optical signals from the optical sensor. For example, the processing of the optical signals can include identifying one or more features of the cardiac cycle in a PPG signal (e.g., the systolic peak, diastolic notch, diastolic peak, etc.). The one or more features can be used to identify each cardiac cycle (e.g., those not corrupted by motion artifacts) and a location in time of the cardiac cycle (e.g., corresponding to the timing of one of the features). Additionally, the processing of optical signals by the one or more processors 108 can include computing a confidence parameter associated with each cardiac cycle (e.g., based on the morphology of the PPG signal). In some examples, the processing of optical signals by the one or more processors 108 can include identifying qualifying cardiac cycles (qualifying pulses) in the optical signals using the one or more features for cardiac cycles with a confidence parameter satisfying one or more qualifying criteria. In some examples, a cardiac cycle can be qualifying when the confidence parameter is above a threshold and non-qualifying when the confidence parameter is below a threshold.

The one or more sensors can include a motion and/or orientation sensor such as an accelerometer, a gyroscope, an inertia-measurement unit (IMU), etc. For example, the wearable device 100 can include accelerometer 106 that can be a multi-channel accelerometer (e.g., a 3-axis accelerometer). As described in more detail herein, the motion and/or orientation sensor can be used for hypertension monitoring. In some examples, the motion and/or orientation information can be useful to provide an indication of motion artifacts and/or user pose that may impact (e.g., corrupt) some samples of the PPG signal. Additionally or alternatively, the motion and/or orientation data may also carry information about heartbeats and this information (and its timing relative to the heartbeat in the PPG signal) may be useful for estimating hypertension scores/parameters as described herein. Measurements by accelerometer 106 can be converted to digital signals for processing via an ADC 105a.

The wearable device 100 can also optionally include other sensors including, but not limited to, a photothermal sensor, a magnetometer, a barometer, a compass, a proximity sensor, a camera, an ambient light sensor, a thermometer, a global position system sensor, and various system sensors which can sense remaining battery life, power consumption, processor speed, CPU load, and the like. Although various sensors are described, it is understood that fewer, more or different sensors may be used.

The data acquired from the one or more sensors (e.g., motion data, optical data, etc.) can be stored in memory in wearable device 100. For example, wearable device 100 can include a data buffer (or other volatile or non-volatile memory or storage) to store temporarily (or permanently) the data from the sensors for processing by processing circuitry. In some examples, volatile or non-volatile memory or storage can be used to store processed data (e.g., filtered data, short-term hypertension scores or parameters, long-term hypertension scores, etc.) for further processing or for storage and/or display of hypertension monitoring results. In some examples, the volatile or non-volatile memory or storage can be used to store processed data referred to herein as pulse data indicating locations of qualifying pulses or indicating locations of pulses and confidence parameters associated with the locations of the pulses. Additionally or alternatively, the volatile or non-volatile memory or storage can be used to store processed data referred to herein as extracted feature data including features extracted from the optical data on a per-pulse basis, over form some or all pulses in an input segment (optionally averaging or otherwise aggregating the per-pulse extracted features across some or all pulses in the input segment).

The wearable device 100 can also include processing circuitry to implement the various processing described herein, including generating hypertension scores/parameters and estimating hypertension. The processing circuitry can include one or more processors 108. One or more of the processors can include a digital signal processor (DSP) 109, a microprocessor, a central processing unit (CPU), a programmable logic device (PLD), a field programmable logic array (FPGA), and/or the like

In some examples, some of the processing can be performed by a peripheral device 118 in communication with the wearable device. The peripheral device 118 can be a smart phone, media player, tablet computer, desktop computer, laptop computer, data server, cloud storage service, or any other portable or non-portable electronic computing device (including a second wearable device). Wearable device 100 can also include communication circuitry 110 to communicatively couple to the peripheral device 118 via wired or wireless communication links 124. For example, the communication circuitry 110 can include circuitry for one or more wireless communication protocols including cellular, Bluetooth, Wi-Fi, etc.

In some examples, wearable device 100 can include a touch screen 128 to display the hypertension monitoring results (e.g., displaying a notification to seek a medical diagnosis) and/or to receive input from a user. In some examples, touch screen 128 may be replaced by a non-touch sensitive display or the touch and/or display functionality can be implemented in another device. In some examples, wearable device 100 can include a microphone/speaker 122 for audio input/output functionality, haptic circuitry to provide haptic feedback to the user, and/or other sensors and input/output devices. Wearable device 100 can also include an energy storage device (e.g., a battery) to provide a power supply for the components of wearable device 100.

The one or more processors 108 (also referred to herein as processing circuitry) can be connected to program storage 111 and can be configured to (programmed to) to execute instructions stored in program storage 111 (e.g., a non-transitory computer-readable storage medium). The processing circuitry, for example, can provide control and data signals to generate a display image on touch screen 128, such as a display image of a user interface (UI), optionally including results of hypertension monitoring. The processing circuitry can also receive touch input from touch screen 128. The touch input can be used by computer programs stored in program storage 111 to perform actions that can include, but are not limited to, moving an object such as a cursor or pointer, scrolling or panning, adjusting control settings, opening a file or document, viewing a menu, making a selection, executing instructions, operating a peripheral device connected to the host device, answering a telephone call, placing a telephone call, terminating a telephone call, changing the volume or audio settings, storing information related to telephone communications such as addresses, frequently dialed numbers, received calls, missed calls, logging onto a computer or a computer network, permitting authorized individuals access to restricted areas of the computer or computer network, loading a user profile associated with a user's preferred arrangement of the computer desktop, permitting access to web content, launching a particular program, encrypting or decoding a message, and/or the like. The processing circuitry can also perform additional functions that may not be related to touch processing and display. In some examples, processing circuitry can perform some of the signal processing functions (e.g., hypertension monitoring/scoring) described herein.

Note that one or more of the functions described herein, including hypertension monitoring, can be performed by firmware stored in memory or instructions stored in program storage 111 and executed by the processing circuitry. The firmware can also be stored and/or transported within any non-transitory computer-readable storage medium for use by or in connection with an instruction execution system, apparatus, or device, such as a computer-based system, processor-containing system, or other system that can fetch the instructions from the instruction execution system, apparatus, or device and execute the instructions. In the context of this document, a “non-transitory computer-readable storage medium” can be any medium (excluding signals) that can contain or store the program for use by or in connection with the instruction execution system, apparatus, or device. The computer-readable storage medium can include, but is not limited to, an electronic, magnetic, optical, electromagnetic, infrared, or semiconductor system, apparatus or device, a portable computer diskette (magnetic), a random access memory (RAM) (magnetic), a read-only memory (ROM) (magnetic), an erasable programmable read-only memory (EPROM) (magnetic), or flash memory such as compact flash cards, secured digital cards, USB memory devices, memory sticks, and the like.

The firmware can also be propagated within any transport medium for use by or in connection with an instruction execution system, apparatus, or device, such as a computer-based system, processor-containing system, or other system that can fetch the instructions from the instruction execution system, apparatus, or device and execute the instructions. In the context of this document, a “transport medium” can be any medium that can communicate, propagate or transport the program for use by or in connection with the instruction execution system, apparatus, or device. The transport medium can include, but is not limited to, an electronic, magnetic, optical, electromagnetic or infrared wired or wireless propagation medium.

It should be apparent that the architecture shown in FIG. 1B is only one example architecture, and that the wearable device could have more or fewer components than shown, or a different configuration of components. The various components shown in FIG. 1B can be implemented in hardware, software, firmware or any combination thereof, including one or more signal processing and/or application specific integrated circuits. Additionally, the components illustrated in FIG. 1B can be included within a single device, or can be distributed between multiple devices.

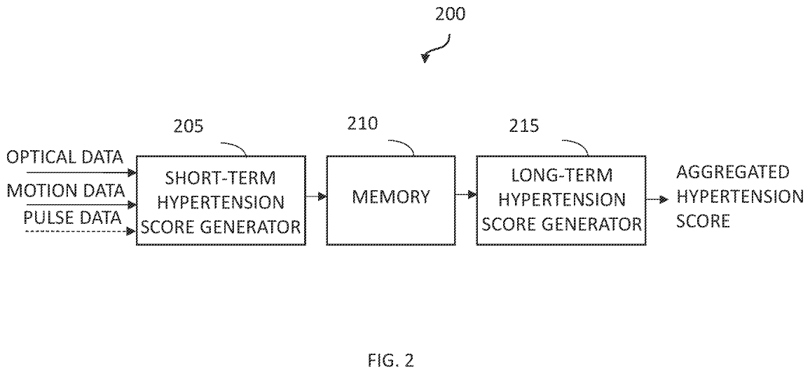

FIG. 2 illustrates an example block diagram for hypertension monitoring according to examples of the disclosure. Block diagram 200 can include processing circuitry (e.g., corresponding to one or more processors 108 and/or DSP 109 in FIG. 1B) to compute hypertension scores and/or parameters. In some examples, the processing circuitry can include a short-term hypertension score generator 205 (a first stage) and a long-term hypertension score generator 215 (a second stage). Block diagram can also include memory 210 that can store short-term hypertension scores/parameters generated by the short-term hypertension score generator 205 and that can be accessed by long-term hypertension score generator 215.

The short-term hypertension score generator 205 can receive, as input, data from the one or more sensors. The data can include optical data from an optical sensor (e.g., a PPG signal) and motion data from a motion sensor (e.g., a three-axis accelerometer). Both the optical data and the motion data can be captured in parallel for a segment of a duration of a first period (e.g., 30 seconds, 1 minute, 2 minutes, 5 minutes, etc.). In some examples, the input to short-term hypertension score generator 205 can also include pulse data indicating the locations of qualifying pulses (or the locations of pulses and confidence parameters associated with the locations of qualifying pulses). The acquisition of the optical data and motion data (and/or the processing of the optical and/or motion data to generate pulse data) can be part of a background process that are executed without a proximate user request to acquire the data. Additionally or alternatively, in some examples, the optical data and the motion data (and/or the processing of the optical and/or motion data to generate pulse data) can be acquired in response to a user request (e.g., a user request to measure heartbeat using the optical and motion sensors). In some examples, the background process to acquire optical data and motion data (and/or the processing of the optical and/or motion data to generate pulse data) can be performed continuously, periodically (e.g., an integer number of times per hour or per day), a threshold period of time after the last measurement of optical/motion data, or in response to various triggers. The frequency of the background process to acquire optical data and the motion data (and/or the processing of the optical and/or motion data to generate pulse data) can be limited, in some examples, as a function of the total power allocated to the background process.

The short-term hypertension score generator 205 can process the segments of optical data and accelerometer data (and optionally pulse data) to generate short-term hypertension scores/parameters that can correlate with the blood pressure. In some examples, the short-term hypertension scores/parameters can include an estimated systolic and diastolic blood pressure (or a systolic score and diastolic score correlating with the systolic and diastolic blood pressure) for each segment. In some examples, the short-term hypertension scores/parameters can include a plurality of parameters (e.g., corresponding to features) extracted from input data rather than a single score for each segment. In some examples, the short-term hypertension scores/parameters can include an estimated overall hypertension score for each segment (without breaking out systolic and diastolic scores). In some examples, the short-term hypertension scores/parameters can be estimated for sub-segments within a segment (e.g., on a per-pulse basis). In some examples, the short-term hypertension scores/parameters for each segment can be stored in memory 210.

In some examples, the optical and motion data (and optionally pulse data) can be processed by the short-term hypertension score generator 205 once data for segment (e.g., for a first period) is acquired (e.g., in response to the acquisition of sufficient optical and motion data for the first period to perform short-term hypertension scoring). In some examples, the segment of optical and motion data (and optionally pulse data) can be stored (e.g., in memory 210 or in a data buffer (not shown)) and can processed later.

The long-term hypertension score generator 215 can process the short-term hypertension scores/parameters (e.g., from memory 210) and can generate a long-term hypertension score that can correlate with the blood pressure and/or a hypertension estimate. The long-term hypertension score can be estimated using an aggregation of short-term hypertension scores/parameters over the course of a second period.

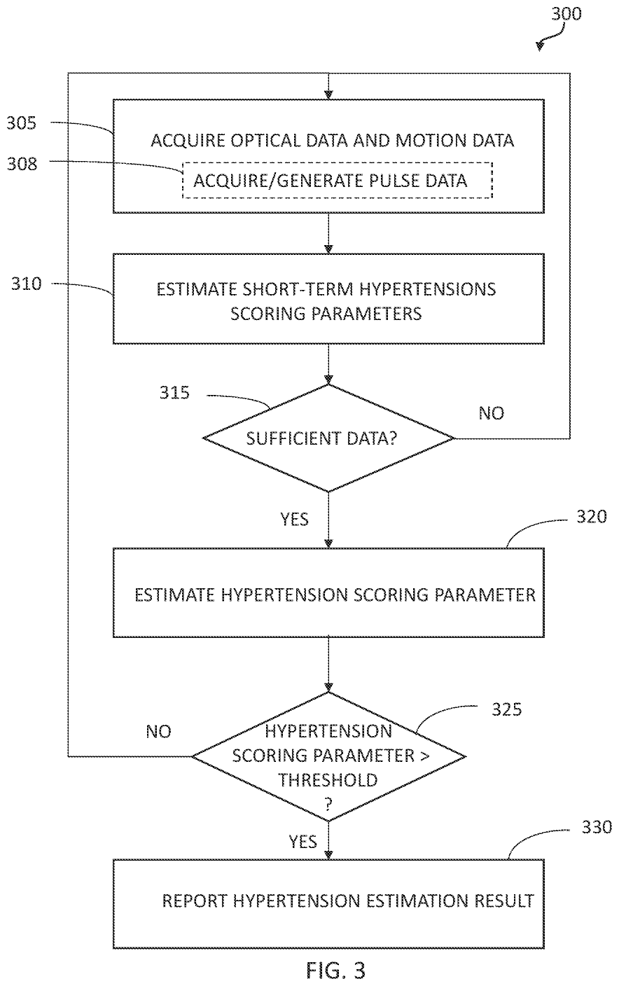

FIG. 3. Illustrates and example process for hypertension monitoring according to examples of the disclosure. Process 300 can be performed by the processing circuitry including processor(s) 108 and/or DSP 109. At 305, optical data and motion data can be acquired for a duration of a first period (e.g., 30 seconds, 1 minute, 2 minutes, 5 minutes, etc.). In some examples, in addition to acquiring optical data and motion data, at 308, pulse data can be acquired (or generated using the optical data and/or motion data) for the segment indicating of qualifying pulses for the segment. The acquisition of the segments of optical data and motion data (and optionally pulse data), can be part of a background process. At 310, short-term hypertension scores/parameters can be generated by short-term hypertension score generator 205 using the segment of optical and motion data (and optionally pulse data). In accordance with a determination that insufficient short-term hypertension scores/parameters are acquired (315), the acquisition of optical and motion data at 305 (and optionally the acquisition of pulse data at 308) and the short-term hypertension scoring at 310 can repeat. In accordance with a determination that sufficient short-term hypertension scores/parameters are acquired (315), a long-term hypertension score can be generated, at 320, by long-term hypertension score generator 215.

In some examples, a determination of the sufficiency or insufficiency of the short-term hypertension scores/parameters can be based on a threshold number of short-term hypertension scores/parameters (e.g., 50, 100, 120, 250, etc.) corresponding to a threshold number of segments of motion and optical data. In some examples, the sufficiency or insufficiency of the short-term hypertension scores/parameters can be based on a period of time. For example, sufficiency of the short-term hypertension scores/parameters can be determined after a threshold period of time, such as the second period (e.g., days, a week, weeks, a month, etc.). In some examples, the sufficiency of the short-term hypertension scores/parameters can be determined by having a threshold number of short-term hypertension scores/parameters for each sub-period of the period of time (e.g., at least one segment used to generate short-term hypertension scores/parameters for each day in the second period).

At 325, the long-term hypertension score/parameter can be thresholded. If the long-term hypertension score/parameter exceeds a threshold, a hypertension estimation result can be reported to the user at 330. For example, a notification can be displayed indicating the possibility of an undiagnosed hypertension condition and/or recommending that a user seek medical care to diagnose hypertension. Additionally or alternatively, in some examples, the user can receive feedback including audio feedback and/or haptic feedback about hypertension monitoring. In some examples, the results can be reported to a health application. In some examples, a notification can be provided to a user's doctor/medical team, if authorized by the user. If the long-term hypertension score/parameter does not exceed the threshold, the user may not be notified of a result. In some examples, the hypertension monitoring process according to process 300 can be performed again (e.g., for the second period) to continue to monitor for hypertension.

The threshold used at 325 can be tuned based on empirical data to reduce a number of false positive results (e.g., false indications of hypertension) and to increase the number of true positive results (e.g., true indications of hypertension). In some examples, the threshold can be tuned to maximize the number of true positive notifications for a stage II hypertension and minimize the number of notifications on non-hypertensive individuals. Although process 300 is described as generating a long-term hypertensive score and using a single threshold to differentiate between hypertensive (and reporting) vs. non-hypertensive (and not reporting) estimates, it should be understood that, in some examples, multiple thresholds can be used to differentiate between multiple levels regarding blood pressure. For example, the thresholding can differentiate between non-hypertensive, elevated blood pressure, stage I hypertensive or stage II hypertensive. In some examples, result can be reported to the user for some of the levels (e.g., stage I and II hypertensive) and not reported for other levels (e.g., non-hypertensive, elevated). In some examples, the specific level can be reported as part of the reporting of the hypertension estimate to the user and/or can be reported to the user's doctor (with or without reporting the specific level to the user).

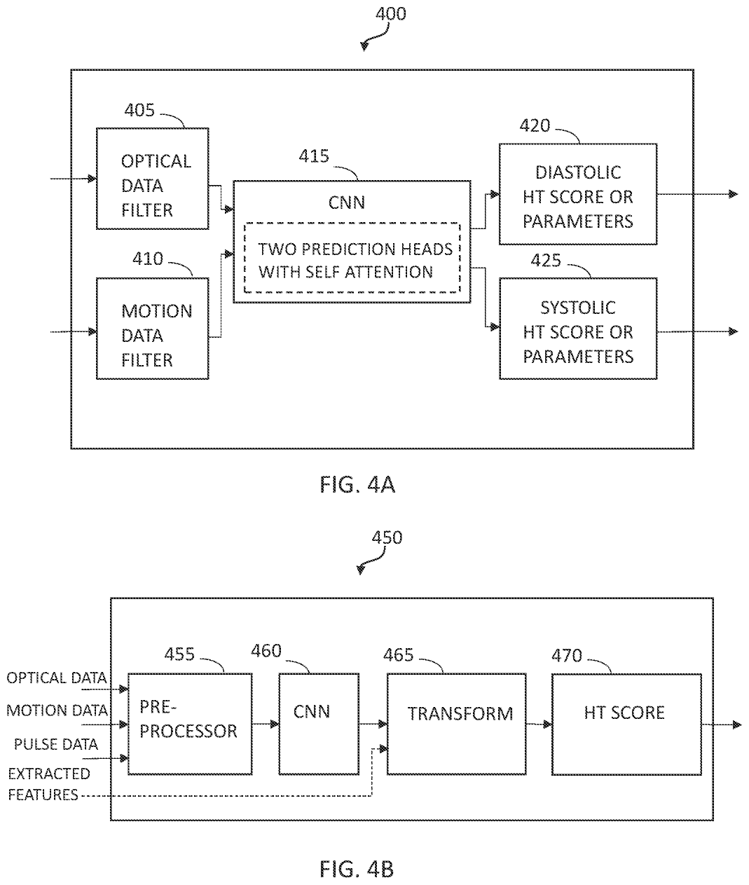

FIGS. 4A-4B illustrate example block diagrams of short-term hypertension score generators according to examples of the disclosure. FIG. 4A illustrates an example block diagram of a short-term hypertension score generator according to examples of the disclosure. Short-term hypertension score generator 400 can correspond to short-term hypertension score generator 205 in FIG. 2. In some examples, short-term hypertension score generator 400 can include an optical data filter 405, a motion data filter 410, and a machine learning processing circuit 415. Optical data filter 405 can band-pass filter the optical data (e.g., passing a range of frequencies such as between 0.1-8 Hz or between 0.5 Hz to 20 Hz) from an optical sensor (e.g., a PPG signal from an optical sensor including one or more light emitters 102 and one or more light sensors 104). Motion data filter 410 can band-pass filter the motion data from a motion sensor (e.g., a multi-axis accelerometer 106).

Machine learning processing circuit 415 can be a two-headed convolutional neural network (CNN) with self-attention. The stem of the CNN can include multiple convolutional layers, which can be organized into residual blocks, in some examples. The CNN can transform the input time-series tensor of filtered optical and motion data (e.g., a segment of the optical/motion data) to extract a set of features (short-term hypertension parameters) for the prediction heads. The prediction heads can then perform computations on these features to generate systolic and diastolic hypertension scores. For example, a first of the prediction head outputs can be a diastolic hypertension score 420 and a second of the prediction head outputs can be a systolic hypertension score 425. These systolic and diastolic hypertension scores can correlate with blood pressure for the sample. In some examples, each prediction head can be embedded with a self-attention mechanism that can enable the respective prediction head to attend to parts of the feature space most salient to its target (e.g., the systolic or the diastolic blood pressure). Both the feature representation (e.g., the set of features) and the self-attention can be learned automatically from labeled training data in an end-to-end fashion. For example, the training data can be acquired by measuring blood pressure with a device (e.g., a blood pressure cuff) to provide labeled systolic and diastolic blood pressure in parallel with (or in proximity to) measuring PPG and accelerometer data via the wearable device. The coefficients of the CNN can be tuned, in some examples, to minimize the mean absolute error (MAE) between the output short-term hypertension scores and the short-term systolic and diastolic blood pressure labels from the training data. In some examples, rather than computing the short-term hypertension scores, the features can be stored as short-term hypertension parameters. The short-term hypertension scores and/or parameters can be stored in memory.

FIG. 4B illustrates another example block diagram of a short-term hypertension score generator according to examples of the disclosure. Short-term hypertension score generator 450 can correspond to short-term hypertension score generator 205 in FIG. 2. In some examples, short-term hypertension score generator 450 can receive filtered optical data, filtered motion data, and pulse data. In some examples, the pulse data can include information about the relative location(s) in time of a pulse or of multiple pulses and/or information regarding the quality of the optical data corresponding to the pulse(s). In some examples, the pulse data can include locations of qualifying pulses (e.g., pulse with a confidence parameter satisfying one or more qualifying criteria). In some examples, the relative location in time of a pulse can be defined by location in time of a specific feature in the morphology of the optical signal, such as a features of the cardiac cycle represented in the optical signal. In some examples, the feature can be a systolic peak, a diastolic notch, or a diastolic peak. In some examples, short-term hypertension score generator 450 can include an optical data filter (e.g., similar to optical data filter 405 described with reference to FIG. 4A, but not shown in FIG. 4B) and/or a motion data filter (e.g., similar to motion data filter 410 described with reference to FIG. 4A, but not shown in FIG. 4B). In some examples, short-term hypertension score generator 450 can also receive extracted features data extracted from the optical data (e.g., frequency, amplitude, phase and/or other timing characteristics of features of the PPG signal). In some examples, the extracted features data can be extracted from the optical data for one or more pulses satisfying the same or similar qualifying criteria (e.g., pulses meeting or exceeding a threshold confidence).

Short-term hypertension score generator 450 can include a pre-processing circuit 455, a machine learning processing circuit 460, and a transformation circuit 465. Pre-processing circuit 455 (also referred to herein as pre-processor) can receive the input time-series tensor of filtered optical and motion data (e.g., a segment of the optical/motion data) and the pulse data to divide the input time-series tensor of filtered optical and motion data into discrete sub-segments (also referred to herein as pulse windows). In some examples, each sub-segment/pulse window can be of the same duration (e.g., 0.5 second, 0.75 seconds, 1 second, etc.), and the sub-segment/pulse window can be defined relative to the location in time of a qualifying pulse indicated by the pulse data. In some examples, the pulse window can be centered on the location in time of the pulse indicated by the pulse data. In some examples, the pulse window can begin at the location in time of the pulse indicated by the pulse data.

In some examples, the number of pulse windows for the input time-series tensor can be capped (e.g., at 50 pulse windows, 60 pulse windows, 70 pulse windows, etc.). In some examples, the number of pulse windows can be capped such that the number of qualifying pulses times the duration of the pulse window is less than or equal to the duration of the input-time series tensor. In some examples, the pulse windows corresponding to pulses with highest confidence are used and those above the maximum number of pulse windows with the lowest confidence can be discarded (not used for short-term hypertension scoring). In some examples, the input time-series tensor is divided into pulse windows sequentially and once the maximum number of pulse widows is achieved, the division of the input time-series tensor can conclude.

In some examples, short-term hypertension scoring can require a minimum number of pulse windows. When the number of pulse windows is less than the minimum number of pulse windows (e.g., as determined as part of the pre-processing), the short-term hypertension score generator can bypass short-term hypertension scoring for the input time-series tensor with fewer than the minimum number of pulse windows. When the number of pulse windows is at or above the minimum number of pulse windows (e.g., as determined as part of the pre-processing), the short-term hypertension score generator can perform the short-term hypertension scoring for the input time-series tensor with at least the minimum number of pulse windows. In some examples, the minimum number of pulse windows can be one pulse window. In some examples, the minimum number of pulse windows can be greater than one pulse window (e.g., 2, 5, 10, etc.).

In some examples, pre-processing circuit 455 can scale the pulse windows. In some examples, the optical data and/or the motion data for each of the pulse windows for qualifying pulses can be scaled by the channel-specific standard deviation (e.g., a first channel of optical data can be scaled by the standard deviation for the time-series input tensor for the first channel). In some examples, the optical data and/or the motion data for each of the pulse windows can be capped at a maximum value (e.g., clipping values with an absolute value greater than 1).

The output of pre-processing circuit 455 (e.g., one or more filtered and scaled pulse windows) can serve as input to a machine learning processing circuit 460. Machine learning processing circuit 460 can be a convolutional neural network (CNN). In some examples, the stem of the CNN can be composed of convolutional layers organized into residual blocks forming the “feature extractor” of the short-term hypertension score generator. The CNN can extract a set of features (short-term hypertension parameters) for each pulse window, which may be referred to herein as a feature vector. In some examples, the CNN can branch into prediction heads to generate feature representations for systolic and diastolic hypertension parameters, which may also be referred to as systolic feature vector and diastolic feature vectors, respectively. In some examples, the systolic and diastolic hypertension parameters/feature vectors can be combined into one set of features (e.g., concatenating the systolic and diastolic hypertension parameters into a single vector).

In some examples, the set of features can be aggregated across the pulse windows in the input (e.g., after generating a set of features of each pulse window in the input segment). In some examples, the aggregation can be an average of each feature in the set of features for each pulse window. In some examples, the aggregation can be an average of each feature in the systolic feature vector across pulse windows in the input segment and an average of each feature in the diastolic feature vector across pulse windows in the input segment.

In some examples, the set of features can branch into prediction heads that perform computations on the sets of features (e.g., the aggregated systolic and diastolic feature vectors) to generate systolic and diastolic hypertension scores that can correlate with blood pressure for the input segment. The systolic and diastolic hypertension scores can be used to generate a short-term hypertension score for the input segment. In some examples, the computations to generate the systolic and diastolic hypertension scores and the short-term hypertension can be achieved using one or more transformations.

For example, as illustrated in FIG. 4B, the feature representation output of CNN 460 can be transformed using transformation circuit 465 to apply one or more transforms to the feature representation to generate short-term hypertension score 470. Short-term hypertension score 470 can be stored in memory. In some examples, transformation circuit 465 can apply one or more linear transformation to convert a high dimensional feature vector output by CNN 460 into a scalar-valued short-term hypertension score. For example, the linear transform can rotate the feature representation vector to a new basis. For example, one linear transform can be used to transform the feature representation into an orthonormal representation that is mutually independent and sorted according to significance (e.g., how much variance in the hypertension result that the feature accounts for). For example, principal component analysis (PCA) can be applied to training data to learn a change-of-basis matrix W_{PCA} that rotates the feature vector into an orthonormal representation. One or more additional linear transformations can be applied to predict long-term systolic and diastolic pressure scores and/or a long-term hypertension score. For example, a first multi-output ridge-regression (e.g., using L2 regularization) can be applied to training data to predict long-term systolic and diastolic blood pressure scores for the new basis. A second ridge-regression (e.g., using L2 regularization) can be applied to training data to predict long-term hypertension status/score from the predicted systolic and diastolic blood pressure scores. The resulting regression weights from the first ridge-regression and the second ridge-regression can learn matrix W_{BP} (systolic/diastolic blood pressure weights) and matrix W_{HT} (hypertension weights).

In some examples, the linear transformations described above can be applied in multiple operations. For example, a first transform can be applied to the short term feature vector using matrix W_{PCA} to change basis, a second transform can then be applied to the feature vector in the new basis using W_{BP} to predict systolic and diastolic blood pressure scores, and a third transform can then be applied to the predicted systolic and diastolic blood pressure scores using W_{HT} to predict a short-term hypertension score (e.g., a scalar value). In some examples, some or all of the transforms can be combined and applied in fewer steps or a single step. For example, a single omnibus weight matrix W_{O} can be applied to the feature vector output by CNN 460 in one transformation operation, where W_{O}=W_{PCA}*W_{BP}*W_{HT}. The single omnibus weight can reduce storage requirements (one matrix rather than three) and reduce processing time/operations (one transformation rather than three).

In some examples, the extracted features data can be combined with the systolic and diastolic hypertension parameters/feature vectors can be into a combined feature set (e.g., concatenating the extracted feature data with the systolic and diastolic hypertension parameters/vectors into a single vector). In some examples, the combination can occur prior to performing any of the transformations. In some examples, the combination can occur after applying a first transform (e.g., the first linear transform to change basis), and the subsequent transformations (e.g., the second and third linear transforms) can be applied to the combined feature set. The extracted features data can refine the short-term hypertension scoring and provide for improved accuracy of hypertension estimation.

Although aggregation of the set of features across pulses described above applies to the systolic and diastolic feature vectors, it should be understood that aggregation can be applied at a different stage of processing. In some examples, the aggregation can apply earlier in the processing to the full set of features before branching into separate systolic and diastolic heads. In some examples, the systolic and diastolic feature vectors described above can be used to compute systolic and diastolic hypertension scores in the systolic and diastolic heads for each pulse, and then the systolic and diastolic hypertension scores can be aggregated across each pulse window in the input segment.

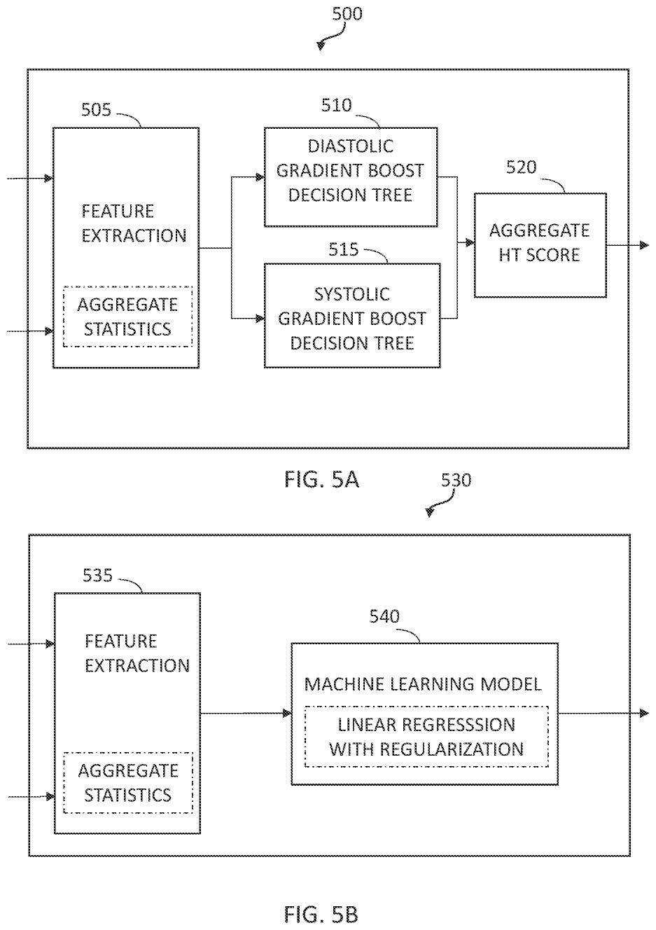

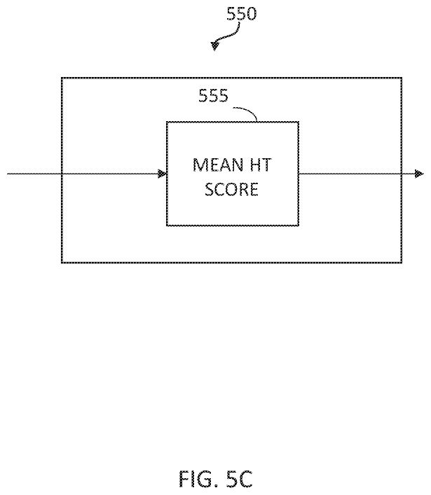

FIGS. 5A-5C illustrate example block diagrams of long-term hypertension score generators according to examples of the disclosure. Long-term hypertension score generator 500, long-term hypertension score generator 530, or long-term hypertension score generator 550 can correspond to long-term hypertension score generator 215 in FIG. 2. It should be understood that long-term hypertension score generators 500, 530 and 550 are example implementations, and that other implementations are possible. More generally, the long-term hypertension score generator can receive short-term hypertension scores/parameters and output a long-term hypertension score (e.g., using aggregation of the short-term hypertension scores/parameters and/or using a machine learning model).

Referring to FIG. 5A, long-term hypertension score generator 500 can include feature extraction block 505, a diastolic decision tree 510 and a systolic decision tree 515. Long-term hypertension score generator 500 can be used to generate a single, long-term hypertension score using aggregated short-term hypertension scores (e.g., a variable length time series of short-term hypertension scores) output by short-term hypertension score generator 400.

Feature extraction block 505 can receive short-term hypertension scores (e.g., stored in memory 210) as inputs. In some examples, feature extraction block 505 can extract statistical features from the aggregated short-term hypertension scores. For example, the distribution of hypertension scores can be summarized by some or all of the mean, median, mode, variance, and/or percentiles, among other possible aggregate statistical measures.

Diastolic decision tree 510 and systolic decision tree 515 can each be gradient-boosted decision tree machine learning models. The diastolic decision tree 510 and the systolic decision tree 515 can each receive the output of feature extraction block 505 as input, and can output a long-term systolic hypertension score (that correlates with aggregate systolic blood pressure) and a long-term diastolic hypertension score (that correlates with aggregate diastolic blood pressure). The decision trees can be trained using the short-term hypertension scores and long-term, user-level blood pressure labels. The training can work to minimize the MAE between long-term, user-level blood pressure labels and the output of the decision trees. In some examples, to prevent overfitting, the number of trees that the gradient-boosted decision tree may learn may be limited (e.g., based on the error measures on a validation data set similar to, but separate from, the training data). The gradient-boosted decision trees can learn different weighting parameters for the input features (with each successive tree in the sequence correcting mistakes by predecessor tree(s) by applying different weights) such that the ensemble of decision trees provides a non-linear predictive function.

In some examples, as illustrated in FIG. 5A, the long-term hypertension score generator 500 can include separate gradient-boosted decision trees to take advantage of feature combinations that may be unique to systolic or diastolic blood pressure. The result can then be aggregated (e.g., using a weighted average) into a single long-term hypertension score 520 that can be used to estimate hypertension and/or report hypertension when the long-term hypertension score is above a threshold (e.g., as described with respect to process 300). In some examples, rather than using separate systolic and diastolic gradient-boosted decision trees 510 and 515, one set of gradient boosted decision trees can be used to generate the long-term hypertension score 520 (without intervening systolic and diastolic scores).

Referring to FIG. 5B, long-term hypertension score generator 530 can include feature extraction block 535 and a machine learning model 540 (e.g., a linear regression with regularization model). Long-term hypertension score generator 530 can be used to generate a single, long-term hypertension score from aggregating short-term hypertension parameters output by short-term hypertension score generator 400. Feature extraction block 535 can receive short-term hypertension parameters (e.g., stored in memory 210) as inputs.

In some examples, feature extraction block 535 can aggregate short-term hypertension parameters. For example, aggregate statistics can be computed for short-term hypertension parameters. In some examples, the aggregate statistics can include a mean vector that be computed to average the sets of short-term hypertension parameters (one set per segment). For example, each estimate of short-term hypertension parameters from short-term hypertension score generator can include N parameters (features) that can be represented in a vector. The N parameters from each of M short-term estimates (for M segments) can be averaged, to result in a mean vector with N parameters (e.g., average parameter 1 from each of the M short-term estimates, average parameter 2 from each of the M short-term estimates, and so on for the N parameters). In some examples, the aggregate statistics can also include a variance or standard deviation for each of the N parameters across the M short-term estimates. In some examples, other aggregate statistics can be calculated.

In some examples, feature extraction block 535 can also compute a covariance (matrix) of the short-term hypertension parameters (e.g., the N parameters from each of M short-term estimates). The covariance matrix can be represented by its eigenvectors and can be used as additional parameters for input into machine learning model 540. In some examples, to reduce the number of input parameters, the covariance can be estimated using a smaller dimensionality and/or the feature(s) can be represented by fewer eigenvectors of the covariance matrix. For example, in some examples, the short-term hypertension parameters can first be sorted based on variance of each of the N parameters (e.g., using principal component analysis) and the covariance can be computed for a subset of the dimensions of the short-term hypertension parameters (<N dimensions for those parameters with the highest variance, or within a range of variances). In some examples, the approximation of the covariance matrix can use a subset of the eigenvectors of the covariance matrix (e.g., one or more eigenvectors). This subset of eigenvectors can be used together with the mean vector as inputs to machine learning model 540.

Machine learning model 540 can be a linear regression machine learning model (e.g., a ridge regression). Machine learning model 540 can each receive the output of feature extraction block 535 as input, and can output a long-term hypertension score. The machine learning model can be trained using the short-term hypertension parameters (and associated extracted features) and long-term, user-level hypertension labels. The training can work to minimize the MAE between long-term, user-level hypertension labels and the output of machine learning model 540.

Referring to FIG. 5C, long-term hypertension score generator 550 can be used to generate a single, long-term hypertension score using aggregated short-term hypertension scores (e.g., a variable length time series of short-term hypertension scores) output by short-term hypertension score generator 450. Long-term hypertension score generator 550 can include mean block 555 to compute an arithmetic mean of the short-term hypertension scores (e.g., output by short-term hypertension score generator 450).

It should be understood that the components of the block diagrams illustrated in FIGS. 4A-5C can be implemented in hardware or software or a combination thereof. Additionally, it should be understood that the block diagrams are examples, and that the implementations may include fewer, more or different blocks. For example, the filters of short-term hypertension score generator 400 can be implemented in a separate part of the system (e.g., the filtered data streams may not be used exclusively for hypertension monitoring). Additionally, it should be understood that different aggregation techniques and/or feature extraction techniques and/or machine learning techniques than shown in FIGS. 5A-5C can be used to generate a long-term hypertension score. For example, the other machine learning models can be used different from the gradient-boosted decision trees of FIG. 5A and the regularized linear regression model of FIG. 5B.

As discussed above, aspects in of the present technology include the gathering and use of physiological information. The technology may be implemented along with technologies that involve gathering personal data that relates to the user's health and/or uniquely identifies or can be used to contact or locate a specific person. Such personal data can include demographic data, date of birth, location-based data, telephone numbers, email addresses, home addresses, and data or records relating to a user's health or level of fitness (e.g., vital signs measurements, medication information, exercise information, etc.).

The present disclosure recognizes that a user's personal data, including physiological information, such as data generated and used by the present technology, can be used to the benefit of users. For example, assessing a user's sleep conditions, heart rate, and/or blood pressure may allow a user to track or otherwise gain insights about their health.

The present disclosure contemplates that the entities responsible for the collection, analysis, disclosure, transfer, storage, or other use of such personal data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should require receipt of the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. The policies and practices may be adapted depending on the geographic region and/or the particular type and nature of personal data being collected and used.

Despite the foregoing, the present disclosure also contemplates embodiments in which users selectively block the collection of, use of, or access to, personal data, including physiological information. For example, a user may be able to disable hardware and/or software elements that collect physiological information. Further, the present disclosure contemplates that hardware and/or software elements can be provided to prevent or block access to personal data that has already been collected. Specifically, users can select to remove, disable, or restrict access to certain health-related applications collecting users' personal health or fitness data.

Therefore, according to the above, some examples of the disclosure are directed to an electronic device. The electronic device can comprise: an optical sensor; a motion sensor; and processing circuitry coupled to the optical sensor and the motion sensor. The processing circuitry can be configured to: generate a plurality of estimates of hypertension scores or parameters, each respective estimate of the plurality of estimates of hypertension scores or parameters using a respective segment of data from the optical sensor and the motion sensor; and generate an aggregated hypertension score using the plurality of estimates. Additionally or alternatively to one or more of the examples disclosed above, in some examples, the processing circuitry can be further configured to: in accordance with the aggregated hypertension score exceeding a threshold, generate a notification about possible hypertension; and in accordance with the aggregated hypertension score failing to exceed the threshold, forgo generating the notification. Additionally or alternatively to one or more of the examples disclosed above, in some examples, the respective segment can correspond to a duration of a first period and the aggregated hypertension score can correspond to a second period greater than the first period. Additionally or alternatively to one or more of the examples disclosed above, in some examples, the processing circuitry can comprise a first machine learning model configured to generate the plurality of estimate of hypertension scores or parameters. Additionally or alternatively to one or more of the examples disclosed above, in some examples, the first machine learning model can comprise a convolutional neural network. Additionally or alternatively to one or more of the examples disclosed above, in some examples, the first machine learning model can comprise a first prediction head configured to generate a systolic hypertension score or parameters and a second prediction head configured to generate a diastolic hypertension score or parameters. Additionally or alternatively to one or more of the examples disclosed above, in some examples, the processing circuitry can comprise a second machine learning model configured to generate the aggregated hypertension score. Additionally or alternatively to one or more of the examples disclosed above, in some examples, the second machine learning model can comprise one or more gradient-boosted decision trees or a regularized linear regression model. Additionally or alternatively to one or more of the examples disclosed above, in some examples, generating the aggregated hypertension score can comprise computing statistical parameters using the plurality of estimates and generating the aggregated hypertension score using the statistical parameters.

Additionally or alternatively to one or more of the examples disclosed above, in some examples, the processing circuitry can be further configured to divide the respective segment of data from the optical sensor and the motion sensor into one or more pulse windows. Additionally or alternatively to one or more of the examples disclosed above, in some examples, the processing circuitry further configured to scale the one or more pulse windows. Additionally or alternatively to one or more of the examples disclosed above, in some examples, the processing circuitry can comprise a machine learning model configured to generate the plurality of estimate of hypertension scores or parameters. Additionally or alternatively to one or more of the examples disclosed above, in some examples, generating the respective estimate of the plurality of estimates of hypertension scores or parameters using the respective segment of data from the optical sensor and the motion sensor can comprise inputting a plurality of the pulse windows into the machine learning model to generate a feature vector of hypertension parameters for each of the plurality of pulse windows and averaging the feature vectors for the plurality of pulse windows to generate an aggregated feature vector for the respective segment. Additionally or alternatively to one or more of the examples disclosed above, in some examples, generating the respective estimate of the plurality of estimates of hypertension scores or parameters using the respective segment of data from the optical sensor and the motion sensor can comprise transforming the aggregated feature vector for the respective segment to generate the respective estimate with a scalar value. Additionally or alternatively to one or more of the examples disclosed above, in some examples, transforming the aggregated feature vector can comprise applying one or more linear transforms. Additionally or alternatively to one or more of the examples disclosed above, in some examples, the one or more linear transforms can include a transform to change a basis of the aggregated feature vector for the respective segment to a new basis. Additionally or alternatively to one or more of the examples disclosed above, in some examples, the one or more linear transforms can include a transform to predict a systolic hypertension score or parameters and a diastolic hypertension score or parameters from the aggregated feature vector for the respective segment in the new basis. Additionally or alternatively to one or more of the examples disclosed above, in some examples, the one or more linear transforms can include a transform to predict the respective estimate of the hypertension score from the systolic hypertension score or parameters and the diastolic hypertension score or parameters. Additionally or alternatively to one or more of the examples disclosed above, in some examples, generating the aggregated hypertension score comprises averaging the plurality of estimates to generate the aggregated hypertension score. Additionally or alternatively to one or more of the examples disclosed above, in some examples, features extracted from the optical data can be added to the aggregated feature vector before or during the one or more linear transforms.

Some examples of the disclosure are directed to a method. The method can comprise: generating a plurality of estimates of hypertension scores or parameters, each respective estimate of the plurality of estimates of hypertension scores or parameters using a respective segment of data from an optical sensor and a motion sensor; and generating an aggregated hypertension score using the plurality of estimates. Additionally or alternatively to one or more of the examples disclosed above, in some examples, the method can further comprise: in accordance with the aggregated hypertension score exceeding a threshold, generating a notification about possible hypertension; and in accordance with the aggregated hypertension score failing to exceed the threshold, forgoing generating the notification. Additionally or alternatively to one or more of the examples disclosed above, in some examples, the respective segment can correspond to a duration of a first period and the aggregated hypertension score can correspond to a second period greater than the first period. Additionally or alternatively to one or more of the examples disclosed above, in some examples, generating the plurality of estimate of hypertension scores or parameters can comprise applying a first machine learning model to a plurality of segments of data from the optical sensor and the motion sensor. Additionally or alternatively to one or more of the examples disclosed above, in some examples, the first machine learning model can comprise a convolutional neural network. Additionally or alternatively to one or more of the examples disclosed above, in some examples, the first machine learning model can comprise a first prediction head configured to generate a systolic hypertension score or parameters and a second prediction head configured to generate a diastolic hypertension score or parameters. Additionally or alternatively to one or more of the examples disclosed above, in some examples, generating the aggregated hypertension score can comprise applying a second machine learning model to the plurality of estimates. Additionally or alternatively to one or more of the examples disclosed above, in some examples, the second machine learning model can comprise one or more gradient-boosted decision trees or a regularized linear regression model. Additionally or alternatively to one or more of the examples disclosed above, in some examples, generating the aggregated hypertension score can comprise computing statistical parameters using the plurality of estimates and generating the aggregated hypertension score using the statistical parameters.

Additionally or alternatively to one or more of the examples disclosed above, in some examples, the method can further comprise dividing the respective segment of data from the optical sensor and the motion sensor into one or more pulse windows. Additionally or alternatively to one or more of the examples disclosed above, in some examples, the method can further comprise scaling the one or more pulse windows. Additionally or alternatively to one or more of the examples disclosed above, in some examples, generating the plurality of estimate of hypertension scores or parameters can comprise applying a machine learning model configured to generate the plurality of estimate of hypertension scores or parameters. Additionally or alternatively to one or more of the examples disclosed above, in some examples, generating the respective estimate of the plurality of estimates of hypertension scores or parameters using the respective segment of data from the optical sensor and the motion sensor can comprise inputting a plurality of the pulse windows into the machine learning model to generate a feature vector of hypertension parameters for each of the plurality of pulse windows and averaging the feature vectors for the plurality of pulse windows to generate an aggregated feature vector for the respective segment. Additionally or alternatively to one or more of the examples disclosed above, in some examples, generating the respective estimate of the plurality of estimates of hypertension scores or parameters using the respective segment of data from the optical sensor and the motion sensor can comprise transforming the aggregated feature vector for the respective segment to generate the respective estimate with a scalar value. Additionally or alternatively to one or more of the examples disclosed above, in some examples, transforming the aggregated feature vector can comprise applying one or more linear transforms. Additionally or alternatively to one or more of the examples disclosed above, in some examples, the one or more linear transforms can include a transform to change a basis of the aggregated feature vector for the respective segment to a new basis. Additionally or alternatively to one or more of the examples disclosed above, in some examples, the one or more linear transforms can include a transform to predict a systolic hypertension score or parameters and a diastolic hypertension score or parameters from the aggregated feature vector for the respective segment in the new basis. Additionally or alternatively to one or more of the examples disclosed above, in some examples, the one or more linear transforms can include a transform to predict the respective estimate of the hypertension score from the systolic hypertension score or parameters and the diastolic hypertension score or parameters. Additionally or alternatively to one or more of the examples disclosed above, in some examples, generating the aggregated hypertension score comprises averaging the plurality of estimates to generate the aggregated hypertension score. Additionally or alternatively to one or more of the examples disclosed above, in some examples, features extracted from the optical data can be added to the aggregated feature vector before or during the one or more linear transforms.

Some examples of the disclosure are directed to a non-transitory computer readable storage medium. The non-transitory computer readable storage medium can store instructions, which when executed by a device comprising processing circuitry, can cause the processing circuitry to perform any of the above methods.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. An electronic device comprising:
an optical sensor;
a motion sensor; and
processing circuitry coupled to the optical sensor and the motion sensor, the processing circuitry configured to:
generate a plurality of estimates of hypertension scores or parameters, each respective estimate of the plurality of estimates of hypertension scores or parameters using a respective segment of data from the optical sensor and the motion sensor; and
generate an aggregated hypertension score using the plurality of estimates.

2. The electronic device of claim 1, the processing circuitry further configured to:
in accordance with the aggregated hypertension score exceeding a threshold, generate a notification about possible hypertension; and
in accordance with the aggregated hypertension score failing to exceed the threshold, forgo generating the notification.

3. The electronic device of claim 1, wherein the respective segment corresponds to a duration of a first period and the aggregated hypertension score corresponds to a second period greater than the first period.

4. The electronic device of claim 1, wherein the processing circuitry comprises a first machine learning model configured to generate the plurality of estimate of hypertension scores or parameters.

5. The electronic device of claim 4, wherein the first machine learning model comprises a first prediction head configured to generate a systolic hypertension score or parameters and a second prediction head configured to generate a diastolic hypertension score or parameters.

6. The electronic device of claim 4, wherein the processing circuitry comprises a second machine learning model configured to generate the aggregated hypertension score.

7. The electronic device of claim 6, wherein the second machine learning model comprises one or more gradient-boosted decision trees or a regularized linear regression model.

8. The electronic device of claim 1, wherein generating the aggregated hypertension score comprises computing statistical parameters using the plurality of estimates and generating the aggregated hypertension score using the statistical parameters.

9. The electronic device of claim 1, the processing circuitry further configured to:
divide the respective segment of data from the optical sensor and the motion sensor into one or more pulse windows.

10. The electronic device of claim 9, the processing circuitry further configured to:
scale the one or more pulse windows.

11. The electronic device of claim 9, wherein the processing circuitry comprises a machine learning model configured to generate the plurality of estimate of hypertension scores or parameters.

12. The electronic device of claim 11, wherein generating the respective estimate of the plurality of estimates of hypertension scores or parameters using the respective segment of data from the optical sensor and the motion sensor comprises:
inputting a plurality of the pulse windows into the machine learning model to generate a feature vector of hypertension parameters for each of the plurality of pulse windows; and
averaging the feature vectors for the plurality of pulse windows to generate an aggregated feature vector for the respective segment.

13. The electronic device of claim 12, wherein generating the respective estimate of the plurality of estimates of hypertension scores or parameters using the respective segment of data from the optical sensor and the motion sensor comprises:
transforming the aggregated feature vector for the respective segment to generate the respective estimate with a scalar value.

14. The electronic device of claim 13, wherein transforming the aggregated feature vector comprises applying one or more linear transforms.

15. The electronic device of claim 14, wherein the one or more linear transforms includes a transform to change a basis of the aggregated feature vector for the respective segment to a new basis.

16. The electronic device of claim 15, wherein the one or more linear transforms includes a transform to predict a systolic hypertension score or parameters and a diastolic hypertension score or parameters from the aggregated feature vector for the respective segment in the new basis.

17. The electronic device of claim 16, wherein the one or more linear transforms includes a transform to predict the respective estimate of the hypertension score from the systolic hypertension score or parameters and the diastolic hypertension score or parameters.

18. The electronic device of claim 1, wherein generating the aggregated hypertension score comprises averaging the plurality of estimates to generate the aggregated hypertension score.

19. A method comprising:
generating a plurality of estimates of hypertension scores or parameters, each respective estimate of the plurality of estimates of hypertension scores or parameters using a respective segment of data from an optical sensor and a motion sensor; and
generating an aggregated hypertension score using the plurality of estimates.

20. A non-transitory computer readable storage medium storing instructions, which when executed by a device comprising processing circuitry, cause the processing circuitry to:
generate a plurality of estimates of hypertension scores or parameters, each respective estimate of the plurality of estimates of hypertension scores or parameters using a respective segment of data from an optical sensor and a motion sensor; and
generate an aggregated hypertension score using the plurality of estimates.
